(19) United States
(12) Patent Application Publication
(10) Pub. No.: US 2009/0088484 A1
(43) Pub. Date: Apr. 2, 2009

(54) NITRIC OXIDE (NO)-RELEASING POLYMERS AND COMPOUNDS AND USES THEREOF

(76) Inventor: Dillip K. Mohanty, Mount Pleasant, MI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/105,869

(22) Filed: Apr. 18, 2008

Related U.S. Application Data
(60) Provisional application No. 60/975,671, filed on Sep. 27, 2007.

(51) Int. Cl. A61K 47/30; C08G 73/02; A61P 43/00

(52) U.S. Cl. 514/772.3; 528/422; 525/540

(57) ABSTRACT

The invention provides compositions that are NO-releasing polymers or low-molecular-weight NO-releasing compounds. The invention also provides the secondary amine polymers that are the precursors as well as the products of the NO-releasing polymers. The invention provides methods of making the secondary amine polymer precursors and the NO-releasing polymers. The invention provides methods for the nitrosation of low-molecular-weight amines and secondary amine polymers to form the corresponding low-molecular-weight NO-releasing compounds and polymers.

### CROSS-REFERENCE TO RELATED APPLICATIONS

This application claims the benefit of priority of U.S. provisional application 60/975,671 filed Sep. 27, 2007, incorporated herein by reference.

### BACKGROUND

Nitric oxide (NO), a simple diatomic molecule, was considered to be a toxic gas as recently as 1987. By 1988, it became apparent that this “noxious” gas is generated in biological systems and functions as a protective and signaling agent in the regulation of blood pressure and clotting, neurotransmission, and immune response. In other words, NO is a powerful signaling and cytotoxic/cytostatic agent found in nearly every tissue including endothelial cells, neural cells, and macrophages.

In mammals, the production of NO is catalyzed by a family of three enzymes, nNO synthase (nNOS), eNOS, and iNOS, and as such, the NO requirements are met in a healthy individual. Problems arise, however, when this fine balance is disturbed. Excess production of NO can lower blood pressure to dangerous levels. NO, being highly reactive, can also damage tissues and is known to cause damage to protein and DNA. To counteract overproduction, there are numerous NO synthase inhibitors. On the other hand, a deficiency of NO can lead to myriad of difficulties including collapsed blood vessels, impotence, respiratory distress, and unwanted blood clotting.

There have been many approaches to providing therapeutic levels of NO to ameliorate one or more of the conditions caused by NO deficiency. These approaches generally seek to increase systemic NO levels. This can be accomplished by either stimulating endogenous NO production or using exogenous NO sources. Methods to regulate endogenous NO release have primarily focused on activation of synthetic pathways by administering NO precursors like L-arginine and/or L-lysine, or increasing expression of nitric oxide synthase (NOS) using gene therapy. However, these methods remain unproven in effectiveness and safety.

Exogenous NO sources, such as pure NO gas, are generally highly toxic, short-lived, and relatively insoluble in physiological fluids. Consequently, systemic exogenous NO delivery has generally been accomplished using organic nitrate and nitrite ester prodrugs in the form of tablets, intravenous suspensions, sprays, and transdermal patches. Drugs capable of producing NO in vivo, including nitroglycerin (GTN), isoamyl nitrate, isosorbide dinitrate, and sodium nitroprusside (SNP), have been developed to relieve angina by dilating blood vessels. These drugs have to be administered with care due to a variety of side effects. Disadvantageously, systemic NO administration can have devastating side effects including hypotension and free radical cell damage.

### BRIEF DESCRIPTION OF THE INVENTION

The invention provides NO-releasing polymers and low-molecular weight NO-releasing compounds and the methods of their making. In accordance with the invention, the problems of systemic NO administration are solved by use of NO-releasing polymers or low-molecular weight NO-releasing compounds, which can provide localized, or site specific, NO delivery, i.e., targeted and controlled delivery of NO to the deficient areas. Polymers embodying the principles of the invention may be made of varied lengths and release times for the controlled release of NO. Upon release of NO, inert non-reactive polymeric amines or soluble secondary amines are formed that are, in fact, the precursors of the NO-releasing polymers or low-molecular weight NO-releasing compounds, i.e., the amines can be re-nitrosated to form the NO-releasing polymers or low-molecular weight NO-releasing compounds.

### BRIEF DESCRIPTION OF THE DRAWINGS

The principles and operation of the invention may be better understood with reference to the accompanying descriptions and the following drawings in which:

FIG. 1 depicts reaction schemes for the formation of polymeric secondary amines of hyperbranched polyethyleneimines and their conversion to NO-releasing polymers utilizing ortho- or para-fluoronitrobenzene or perfluorobenzene;

FIG. 2 depicts reaction schemes for the formation of polymeric secondary amines of polyvinylamines and their conversion to NO-releasing polymers utilizing ortho- or para-fluoronitrobenzene or perfluorobenzene;

FIG. 3 depicts reaction schemes for the formation of polymeric secondary amines of polyallyllamines and their conversion to NO-releasing polymers utilizing ortho- or para-fluoronitrobenzene or perfluorobenzene;

FIG. 4 depicts reaction schemes for the formation of polymeric secondary amines of polypropylenimine tetraamine dendrimers utilizing ortho- or para-fluoronitrobenzene or perfluorobenzene;

FIG. 5 depicts reaction schemes for the formation of polymeric secondary amines of chitosans and their conversion to NO-releasing polysaccharides utilizing ortho- or para-fluoronitrobenzene or perfluorobenzene;

FIGS. 6 A, B, and C depict reaction schemes for formation of polymeric secondary amines and their corresponding NO-releasing polymers as well as low molecular weight soluble amines and the corresponding NO-releasing compounds;

FIG. 7 depicts a graph showing NO release as a function of time for NO-releasing polymers representing different lengths of polyamine 6 (of FIG. 2); and

FIG. 8 shows NO release as a function of time over 270 hours for N-decyl-p-nitrobenzene in phosphate buffer solution (PBS).

FIG. 9 shows NO release as a function of time over 8 hours for N-decyl-p-nitrobenzene in phosphate buffer solution (PBS).

### DETAILED DESCRIPTION

The invention provides novel NO-donating polymers (also referred to as NO-releasing polymers) and low-molecular weight NO-releasing compounds (also referred to as NO-donating compounds), and the inert polymeric amines or soluble amines that are formed upon release of the NO. The NO-donating polymers and low-molecular weight NO-releasing compounds may be referred to, collectively, as NO-donors. The NO-donating polymers include one or more NO-releasing group(s) covalently linked to a polymeric backbone on the one hand and to a nitro or perfluoronitro aryl group on the other, and are designed such that NO is released from the polymers under certain conditions. The low-molecular weight NO-releasing compounds include secondary amines that are designed such that NO is released from the compounds under certain conditions. The invention further provides methods of preparing the novel NO-donating polymers and inert polymeric amines. The polymeric amines, formed by NO release, are extremely inert with properties similar to the noble metals. These polymeric amines are both the precursors of the NO-donating polymers, the amines being nitrosated to form the NO-donating polymers, and the products of NO-release.

The novel NO-donors of the invention are believed to be of value in the treatment of certain medical conditions, such as ischemic heart disease, heart failure, hypertension and other cardiovascular diseases, pulmonary hypertension, urological disorders, blood clotting, blood pressure, and the destruction of cancerous tumor cells, as well as a variety of neurodegenerative diseases, including Parkinson's Disease, Alzheimer's Disease, Huntington Disease, multiple sclerosis, and other related disorders. The invention also contemplates pharmaceutical compositions including the NO-donors, as well as medical devices designed for various modes of delivering the NO-donating polymers.

Before explaining at least one embodiment of the invention in detail, it is to be understood that the invention is not limited in its application to the details set forth in the following description or exemplified by the Examples, and such description and Examples are not intended to limit the scope of the invention as set forth in the appended claims. The invention is capable of other embodiments or of being practiced or carried out in various ways. For example, while the following detailed description describes the invention through reference to embodiments utilizing certain NO-donating polymers and inert secondary amine polymers, it should be understood that other NO-donating polymers are also suitable for use with the teachings of the invention. Although methods and materials similar or equivalent to those described herein can be used in the practice or testing of the invention, suitable methods and materials are described below. Also, it is to be understood that the phraseology and terminology employed herein is for the purpose of description and should not be regarded as limiting.

Further, the use of “comprising,” “including,” “having,” and variations thereof herein is meant to encompass the items listed thereafter and equivalents thereof as well as additional items, e.g., that other steps and ingredients that do not affect the final result can be added. This term encompasses the terms “consisting of” and “consisting essentially of”. The use of “consisting essentially of” means that the composition or method may include additional ingredients and/or steps, but only if the additional ingredients and/or steps do not materially alter the basic and novel characteristics of the claimed composition or method.

Further, no admission is made that any reference, including any patent or patent document, cited in this specification constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinency of any of the documents cited herein.

Throughout this disclosure, various aspects of this invention may be presented in a range format. It should be understood that the description in range format is merely for convenience and brevity, and should not be construed as an inflexible limitation on the scope of the invention. Accordingly, as will be understood by one skilled in the art, for any and all purposes, particularly in terms of providing a written description, all ranges disclosed herein also encompass any and all possible subranges and combinations of subranges thereof, as well as all integral and fractional numerical values within that range. As only one example, a range of 20% to 40% can be broken down into ranges of 20% to 32.5% and 32.5% to 40%, 20% to 27.5% and 27.5% to 40%, etc. For further example, if a polymer is stated as having 7 to 300 linked monomers, it is intended that values such as 7 to 25, 8 to 30, 9 to 90, or 50 to 300, as well as individual numbers within that range, for example, 25, 50, and 300, are expressly enumerated in this specification. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. Further, as will also be understood by one skilled in the art, all language such as “up to,” “at least,” “greater than,” “less than,” “more than,” and the like include the number recited and refer to ranges which can be subsequently broken down into subranges as discussed above. In the same manner, all ratios disclosed herein also include all subratios falling within the broader ratio. These are only examples of what is specifically intended. Further, the phrases “ranging/ranges between” a first indicate number and a second indicate number and “ranging/ranges from” a first indicate number “to” a second indicate number are used herein interchangeably.

Unless otherwise defined, all technical and scientific terms used herein have the same meaning as commonly understood by one of ordinary skill in the art to which this invention belongs. However, as used herein, the following definitions may be useful in aiding the skilled practitioner in understanding the invention:

As used herein, the phrase “chemical moiety” is meant to refer to a functional group which is a part or component of an organic molecule, and possesses certain chemical properties.

As used herein, “polymer”, “polymeric backbone” or “polymeric species” has the meaning commonly afforded the term. Examples are homopolymers, co-polymers (including block copolymers and graft copolymers), dendritic polymers, crosslinked polymers and the like. Suitable polymers include synthetic and natural polymers (e.g. polysaccharides, polypeptides) as well as polymers prepared by condensation, addition and ring opening polymerizations. Also included are rubbers, fibers and plastics.

In accordance with the invention, a secondary amine group, —NH—, or a N-nitrosamine group, —NNO—, is connected to a polymeric backbone by a single covalent bond between the nitrogen atom of NH or NNO, and is pendant to the polymer. Thus, the polymers of the invention have pendant —NH— groups or —NNO— groups. A polymer with —NH— groups is referred to as a secondary amine polymer. A polymer with a —NNO— group is referred to as a N-nitrosamine polymer or simply, NO-releasing polymer. The pendant secondary amine and N-nitrosamine groups are substituted on a substituted aryl group.

The term “alkyl” refers to a saturated aliphatic hydrocarbon including straight chain and branched chain groups. Suitably, the alkyl group has 1 to 20 carbon atoms. As detailed above, whenever a numerical range, e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted. Substituted alkyl groups may have one or more substituents, wherein each substituent group may independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine, and hydrazine. The alkyl group can be an end group, wherein it is attached to a single adjacent atom, or a linking group, which connects two or more moieties via at least two carbons in its chain.

The term “aryl” refers to an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents in which a suitable substituent group may independently be, for example, nitro, halo, cycloalkyl, alkenyl, alkynyl, aryl, heteroaryl, heteroalicyclic, amine, sulfonate, sulfoxide, phosphonate, hydroxy, alkoxy, aryloxy, thiohydroxy, thioalkoxy, thioaryloxy, cyano, azo, sulfonamide, C-carboxylate, O-carboxylate, N-thiocarbamate, O-thiocarbamate, urea, thiourea, N-carbamate, O-carbamate, C-amide, N-amide, guanyl, guanidine, and hydrazine. The aryl group can be an end group, as this term is defined hereinabove, wherein it is attached to a single adjacent atom, or a linking group, as this term is defined hereinabove, connecting two or more moieties at two or more positions thereof.

The terms “halide” and “halo” refer to fluorine, chlorine, bromine, or iodine.

As used herein, the phrase “NO-releasing group” refers to a chemical moiety, which is capable of generating NO either spontaneously or by means of chemical or enzymatic reactions. In accordance with the invention, a suitable NO-releasing group includes, without limitation, an N-nitrosamine group, —NNO—.

As used herein in connection with numerical values, the terms “about” and “approximately” are meant to encompass variations of about ±20%, including ±10% or less of the indicated value.

The term “treating” includes inhibiting, slowing, or reversing the progression of a medical condition, ameliorating or reducing symptoms of a condition, or preventing the appearance of symptoms of a condition.

The term “active ingredient” refers to a pharmaceutical agent that subsequent to its application has, at the very least, one desired pharmaceutical or therapeutic effect.

The term “therapeutically effective amount” or “pharmaceutically effective amount” denotes that dose of an active ingredient or a composition comprising the active ingredient that will provide the therapeutic effect for which the active ingredient is indicated, i.e., relieving to some extent one or more symptoms of the disease being treated, or modulating, e.g., elevating an NO level.

The term “pharmaceutically acceptable carrier” refers to a carrier or a diluent that does not cause significant irritation to an organism and does not abrogate the biological activity and properties of the active ingredient. Examples, without limitations, of carriers are: propylene glycol, saline, emulsions, and mixtures of organic solvents with water, as well as solid (e.g., powdered) and gaseous carriers.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

As used herein, a “pharmaceutical composition” refers to a preparation of one or more of the NO-donors described herein, with other chemical components such as pharmaceutically acceptable and suitable carriers and excipients. The purpose of a pharmaceutical composition is to facilitate administration of the polymer to an organism.

The term “subject,” as used herein, is intended to describe an animal, more typically a mammal, and most typically a human presenting a physiological disorder or disease for which treatment with NO would be beneficial. In some cases, “subject” refers to an animal that receives a treatment for the purposes of comparison, e.g., a control animal, even though the animal does not present a physiological disorder or disease for which treatment with NO would be beneficial.

As is conventional, the singular form “a,” “an,” and “the” include plural references unless the context clearly dictates otherwise. For example, the term “a compound” or “at least one compound” may include a plurality of compounds, including mixtures thereof.

Organic nitrate and nitrite esters represent a time-honored class of NO-donating agents used in cardiovascular therapeutics since the nineteenth century. These agents have direct vasoactive effects and have therefore been used to treat ischemic heart disease, heart failure, and hypertension for many years. However, as also further discussed hereinabove, treatment methods utilizing these compounds are severely limited by their therapeutic half-life, systemic absorption that is oftentimes accompanied by adverse hemodynamic effects, and drug tolerance.

The invention provides novel NO-donating polymers, which, upon releasing a bioactive NO, an inert polymer is formed. It is contemplated that such NO donors, when entering a biological system, are subjected to enzymatic reactions that result in the release of a bioactive NO and the formation of an inert polymer which would be characterized by non-toxicity and efficient excretion. The invention further provides for NO-releasing, low-molecular-weight, soluble compounds, which upon releasing a bioactive NO, a soluble compound is formed.

It is also envisioned that by conjugating another bioactive or therapeutic compound to the NO-donor, or by co-administering another bioactive or therapeutic agent, combined, and even synergistic, therapeutic effects could be achieved, resulting from the dual therapeutic effect of the bioactive agent and the NO-releasing group.

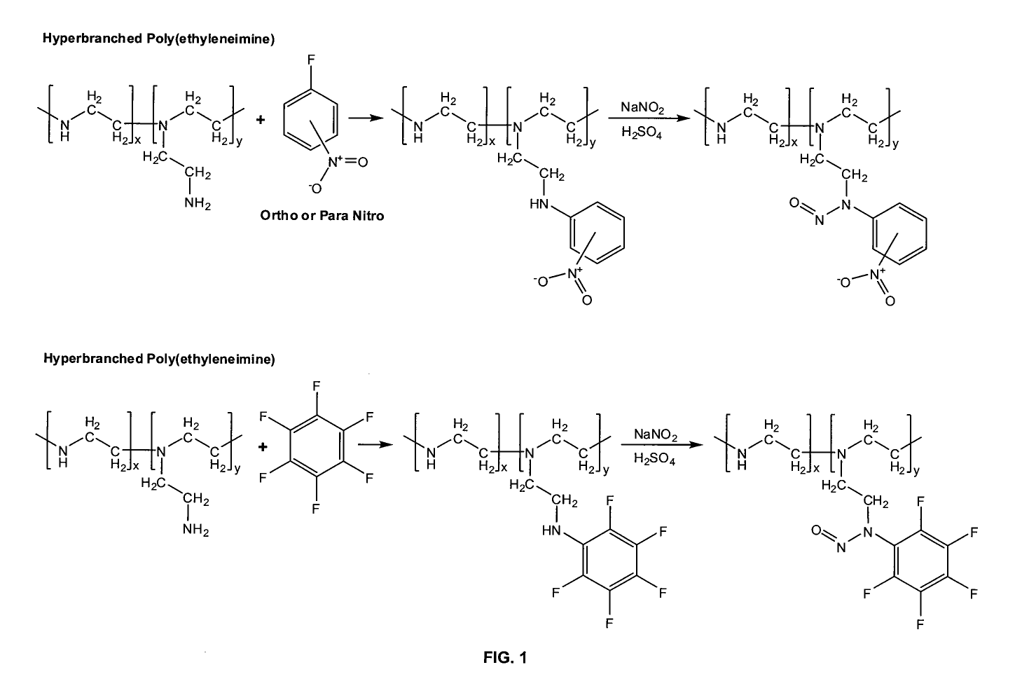

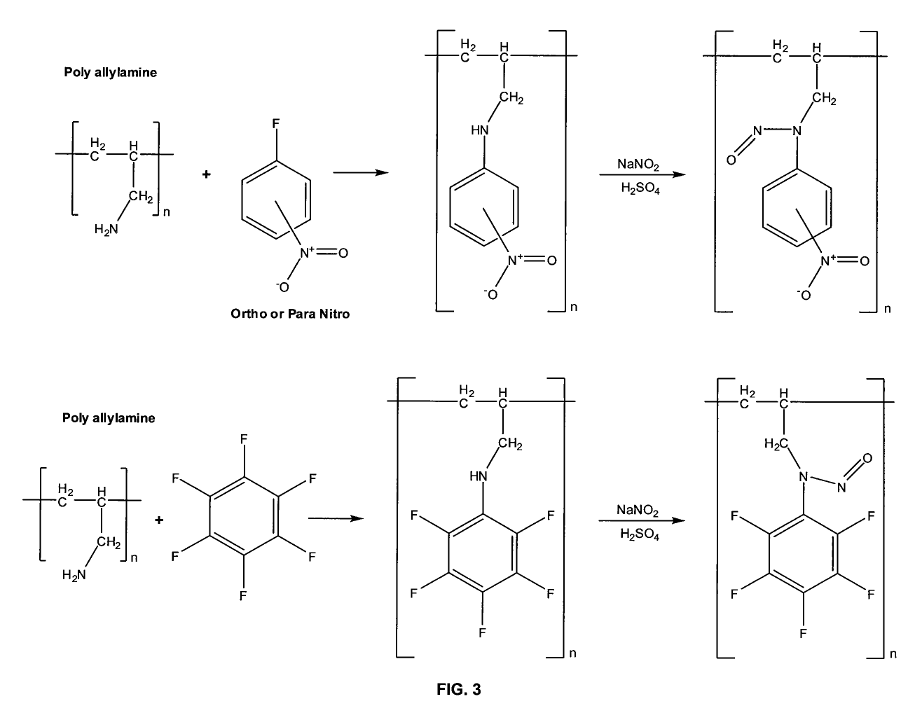

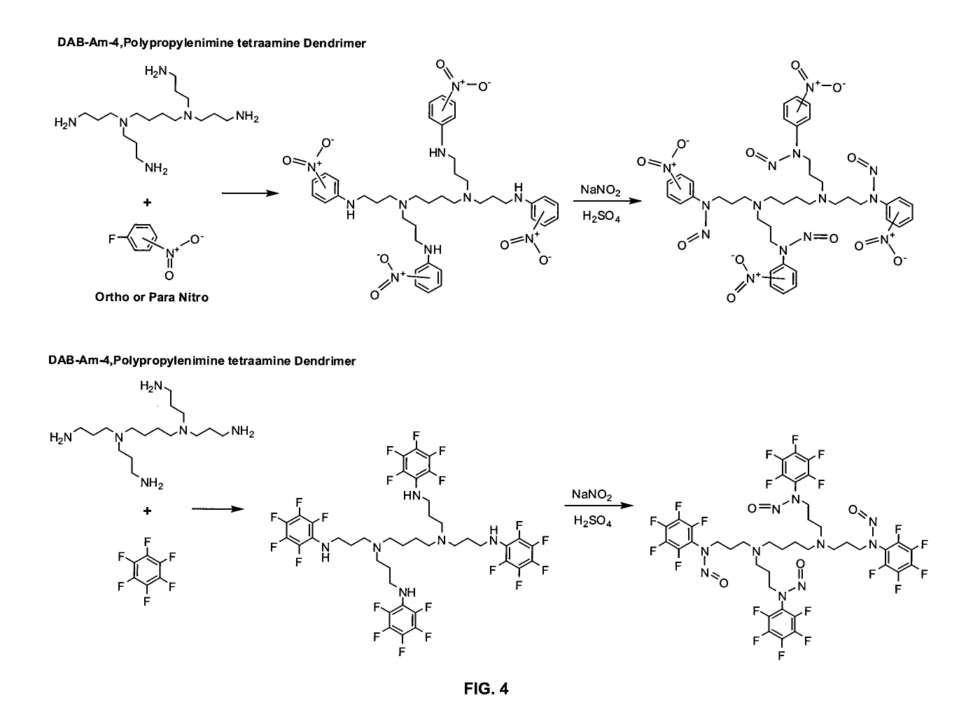

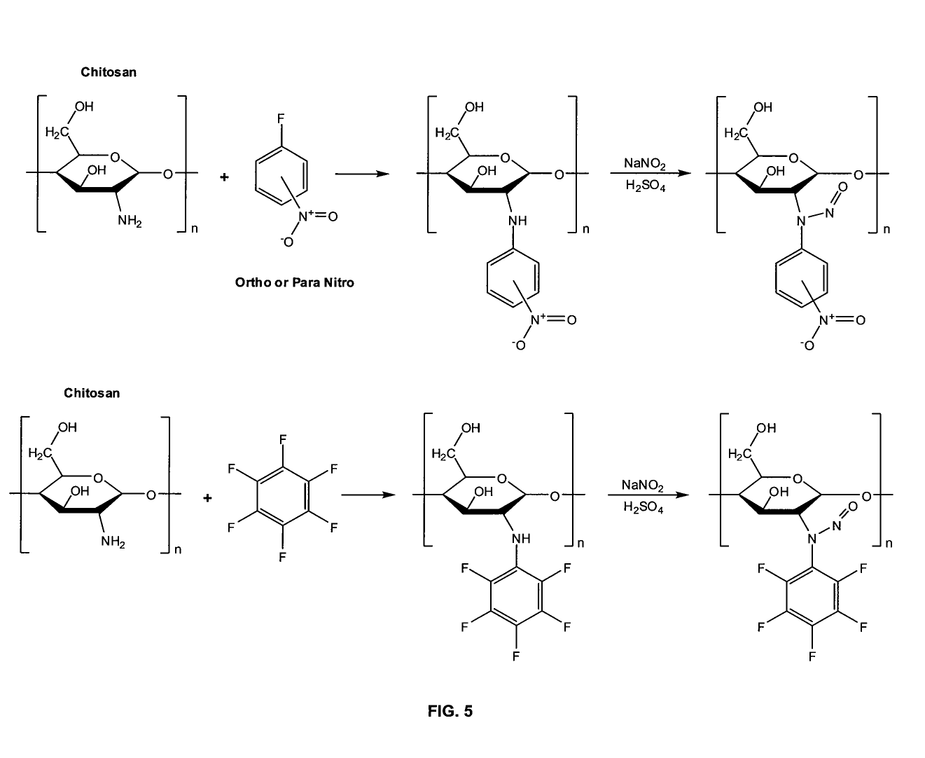

A plurality of NO-donating polymers and the corresponding inert polymeric amines have been synthesized and are shown in FIGS. 1-6. NO-releasing polymers were found to be efficacious in reducing the symptoms of Parkinson's Disease in in vivo models when administered as pellets or pellets within a dialysis bag, as described in the co-pending application entitled “Methods of Treating Disease with Nitric Oxide (NO)-releasing Polymers and Insoluble NO-releasing Nitrosamines,” inventors: J. A. Oh-Lee and D. K. Mohanty.

The invention, therefore, provides novel polymers and compounds capable of delivering NO. Each of the polymers of the invention includes at least one NO-releasing group, as is detailed herein. In one embodiment, the NO-releasing group(s) are covalently linked, on the one hand to the polymer backbone, and to a substituted or unsubstituted aryl group on the other. In another embodiment, the polymer includes at least one dinitroamine monomer, the N-nitrosamine group being linked to a substituted aryl group. In either embodiment, upon release of NO from the polymer, an inert, non-reactive polymer is formed.

The invention further provides a new class of inert polymeric secondary amines. As described above, these secondary amine polymers are both precursors and products of the NO-releasing polymers. This family of polymers exhibits superior mechanical, thermal, hydrolytic, and solvent-resistant properties than conventional polymeric aliphatic secondary amines. These polymeric amines in accordance with the invention have been found to be insoluble in all known organic solvents at room temperature and in most cases under reflux with reflux temperatures approaching 190° C. These secondary amine polymers in accordance with the invention were soluble only in aqueous concentrated mineral acids with bulky counter ions including sulfuric, nitric, and perchloric acids. It is believed that this lack of solubility may be a direct consequence of both inter- and intra-chain H-bonding interactions between the secondary amine groups and the nitro groups present in the repeat unit structure, as shown in FIGS. 1-6. It is also likely that inter-chain π-π interactions between the benzene rings may play a minor role in imparting such excellent solvent resistance characteristics to this family of polymeric amines.

As shown in FIGS. 1-6, the secondary amine group is flanked by an aromatic moiety, which allows for further chemical modifications not feasible with the NH groups of conventional polymeric amines. For example, these NH groups can be nitrosated with ease to produce N-nitrosamines without taking recourse to the use of NO gas under high pressure conditions, as is necessary in the case of aliphatic polymeric secondary amines. This structural feature has been used to prepare N-nitrosamine-containing polymers, i.e., NO-releasing polymers, as shown in FIGS. 1-6. The extent of nitrosation of the polymeric amines in accordance with the invention was determined to be 70% by IR spectroscopy, in sharp contrast to a typical low level (45%) of nitrosation achieved by direct introduction of NO gas under high pressure.

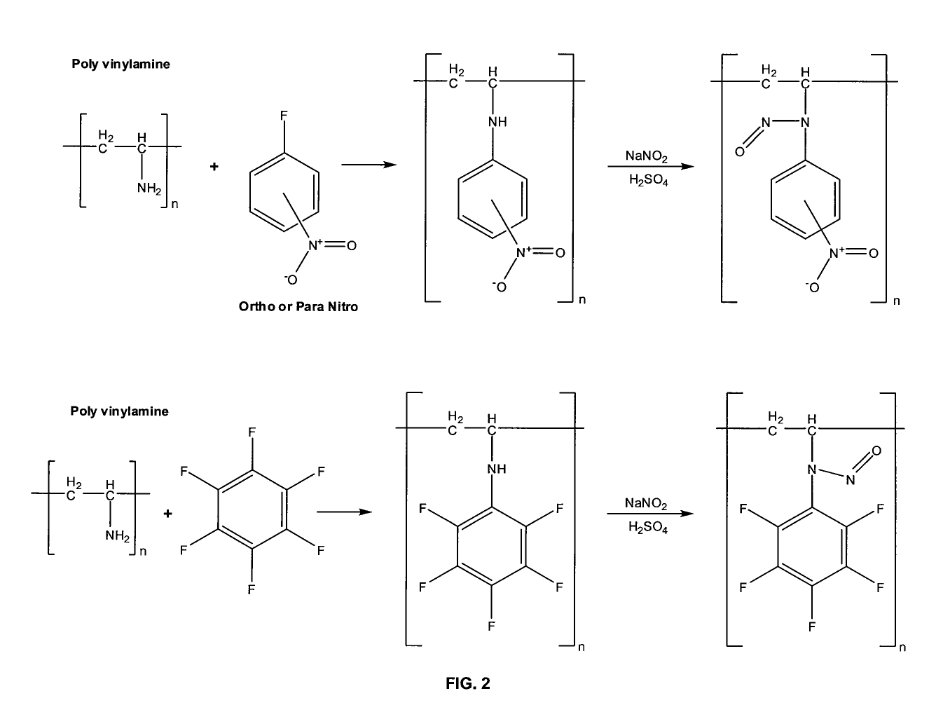

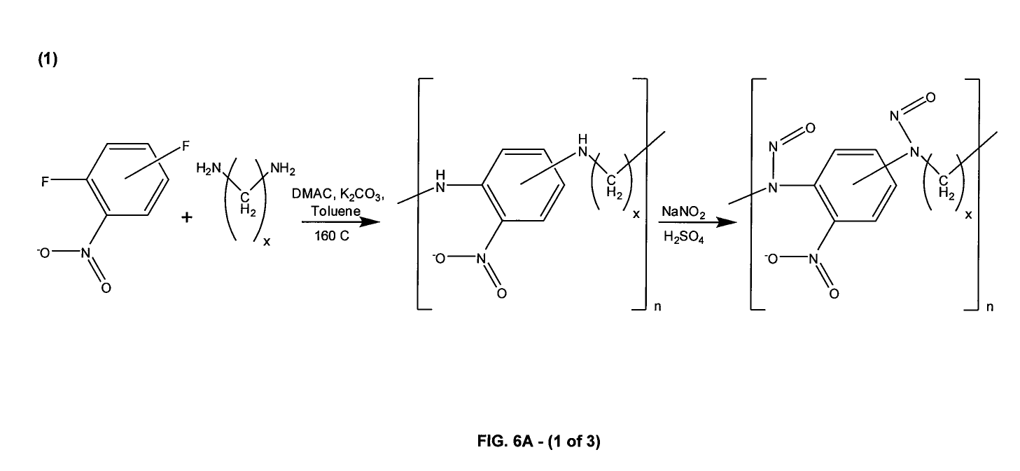

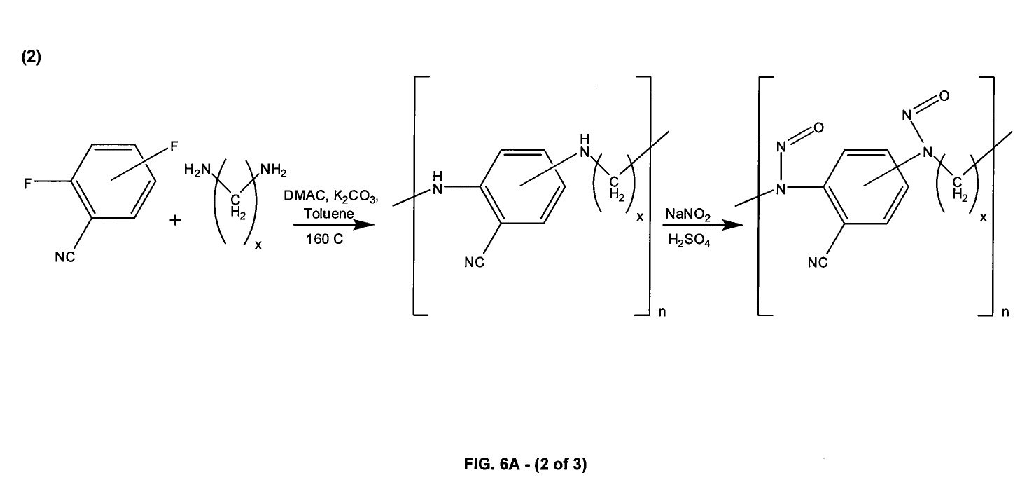

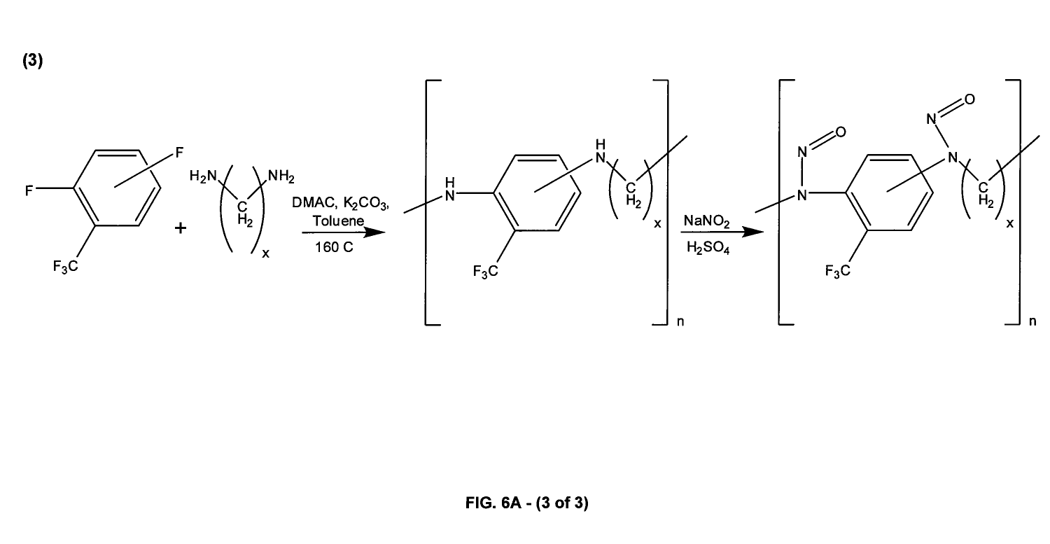

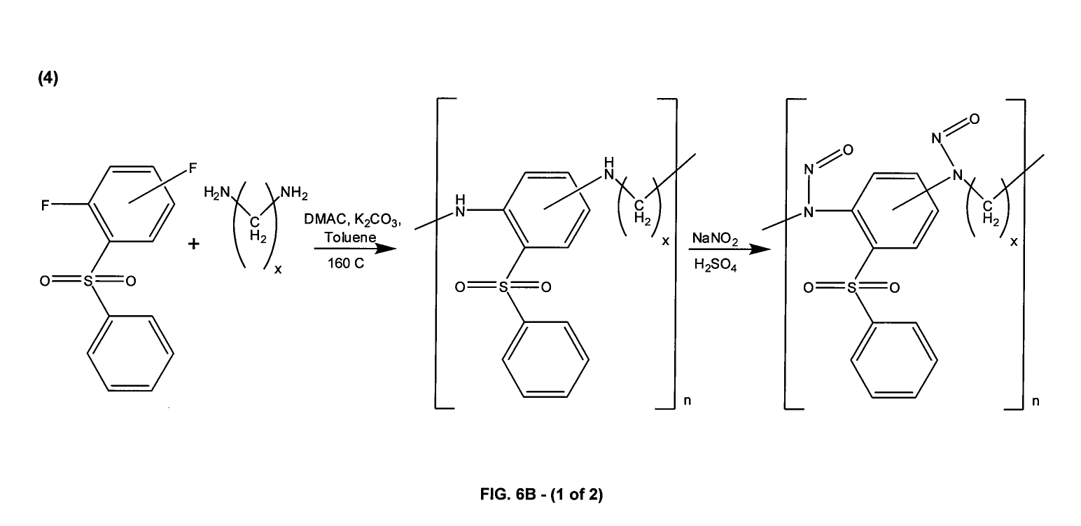

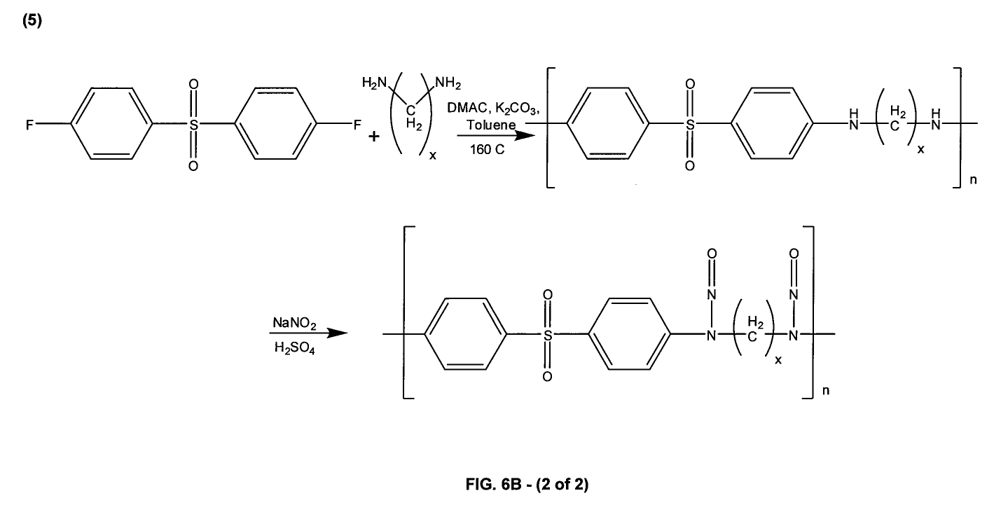

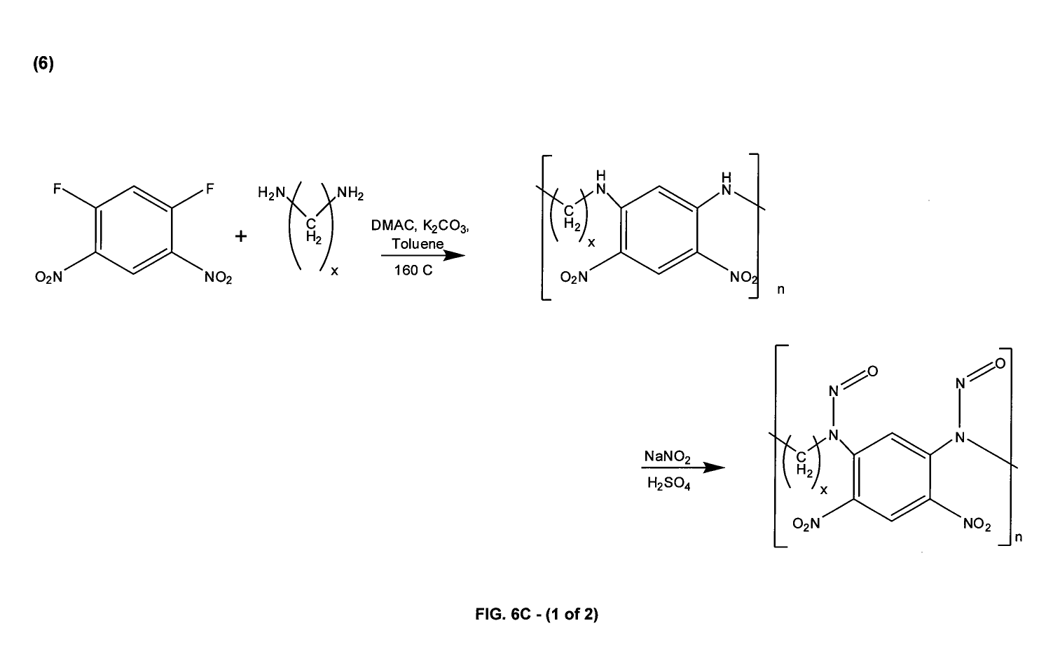

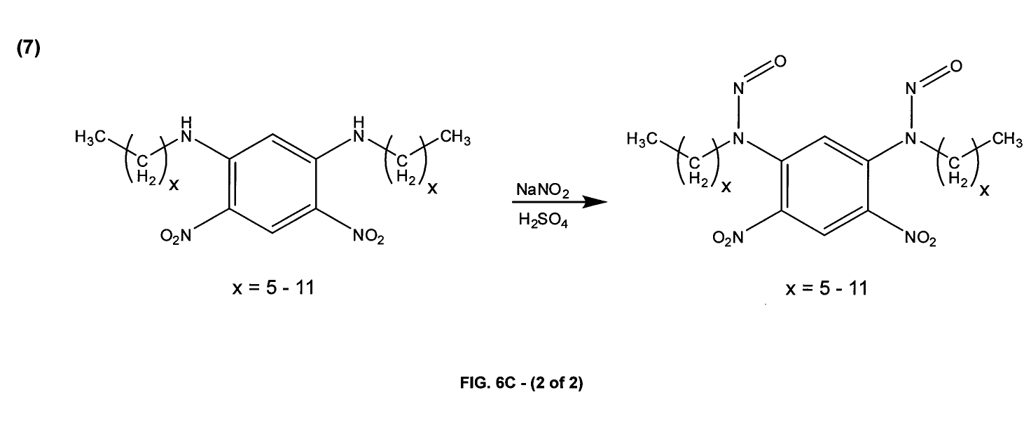

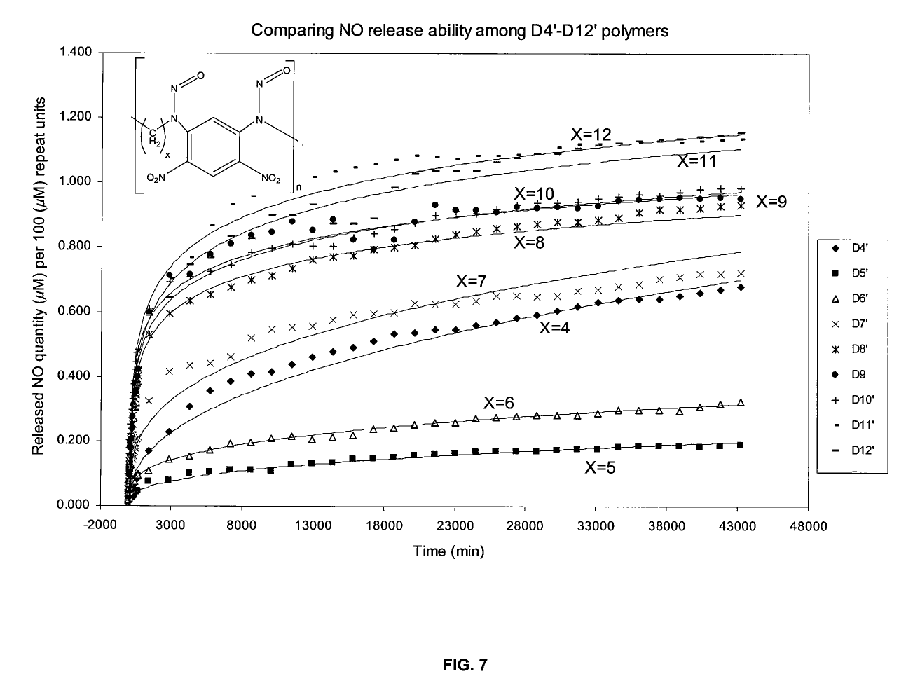

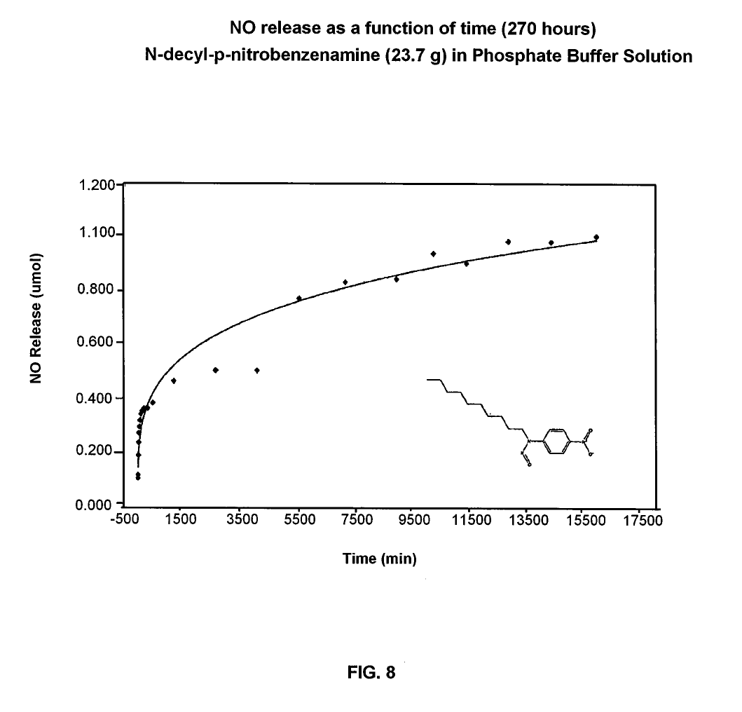

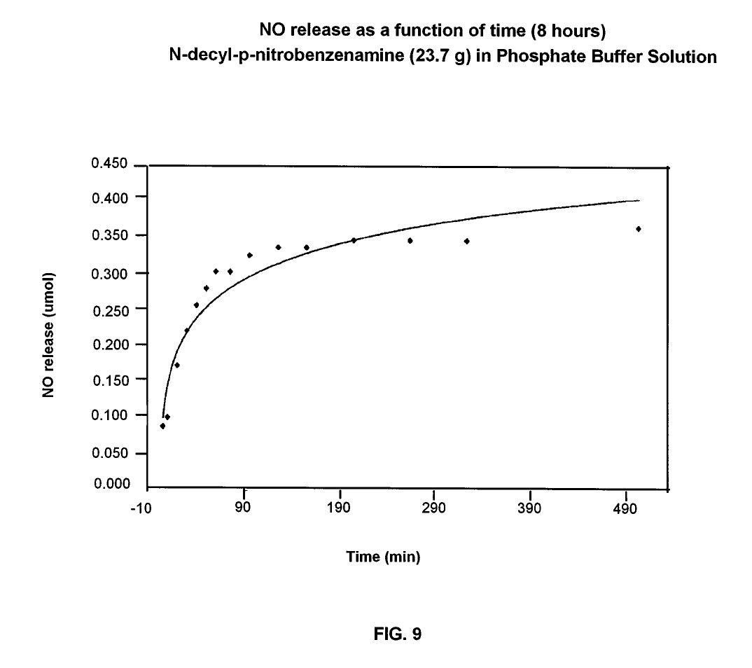

The total amount of NO released from a measured amount of NO-donating polymers, i.e., N-nitrosamine-containing polymers, in accordance with the invention in phosphate buffer solution (PBS), at room temperature, was measured using a colorimetric technique as described in Example 3. NO-release profiles for D4′ to D12′ polymers representing polymer 6 of FIG. 6 are shown in FIG. 7. An examination of the release profile indicates that there is a very rapid onset of NO release, and within ˜200 min. the rate of NO released slows down significantly. These findings show that release of NO continues for at least 30 days. A similar NO release profile was observed when this experiment was conducted in a PBS buffer solution with a model compound representing polymer 1 of FIG. 2, as shown in FIGS. 8-9.

Importantly, the NO-donating polymers are stable when stored at 4° C. under a nitrogen atmosphere. The polymers are temperature-sensitive and decompose when the temperature of a sample is raised to room temperature. Thus, depending on the NO need, NO-releasing polymers, for example, can be implanted near the targeted organ after being brought to room temperature for different lengths of times.

As demonstrated in FIG. 7, the NO-donating polymers of the invention are tunable in that the rate of NO release can be varied based upon the structure of the polymer. The NO-release rate may be varied by incorporating NO-donating monomers with lower NO binding constants, by varying the structure of the intervening alkyl chain between monomer units, and/or by altering the distance between the substituted aryl and the secondary amine which donates the NO.

The NO-donating polymers, in accordance with the invention, after NO release, are converted back to highly insoluble starting polymeric amines. As discussed above, the polymeric amines exhibit a solubility profile typical of noble metals, which in their metallic state are known to be non-toxic. It is envisioned that such stability and lack of solubility may allow for the use of an implantable therapeutic agent which after reacting yields a non-toxic end product, which cannot undergo further metabolism. In addition, an implant (after NO dissipation) may also be removed from the body.

Further according to the invention, there is provided a process of converting the secondary amine polymers or the low molecular-weight soluble compounds into the corresponding NO-releasing polymers and low-molecular weight soluble NO-releasing compounds described hereinabove. As a starting material, a suitable primary amine polymer or low-molecular weight compound is used.

In some embodiments shown in FIGS. 1-5, the polymeric secondary amine precursor/product is represented by general formula (I):

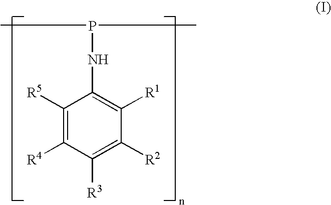

wherein P is a polymeric species with a chemical moiety capable of forming a covalent bond with N, and n is an integer greater than about 20, and R¹, R², R³, R⁴, and R⁵ are H, F, or NO₂ provided that when R¹═F, then R², R³, R⁴, and R⁵═F; and provided that when R¹═NO₂, then R², R³, R⁴, and R⁵═H; provided that when R⁵═NO₂, then R¹, R², R³, and R⁴═H; when R²═NO₂, then R⁴═NO₂ and R¹, R³, and R⁵═H; and when R³═NO₂, then R¹, R², R⁴, and R⁵═H. For those embodiments in which the benzene ring of the polymeric amine is disubstituted with nitro groups, the reagent is 1,5-difluoro-2,4-dinitrobenzene.

The corresponding NO-donor has general formula (II):

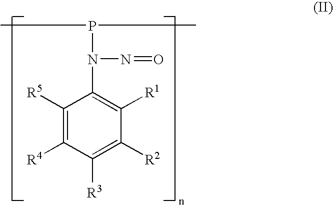

wherein P, n, R¹, R², R³, R⁴, and R⁵ have the same meaning as described above for formula (I).

In other embodiments shown in FIG. 6, the polymeric amine is a polymeric secondary diamine which is represented by general formula (III)

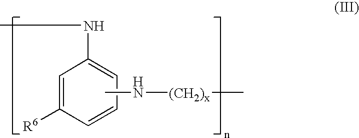

wherein x=2 to 12 and n is an integer greater than about 20, and R⁶ is NO₂, CN, CF₃, or phenyl sulfone.

The corresponding NO-releasing polymer is represented by general formula (IV):

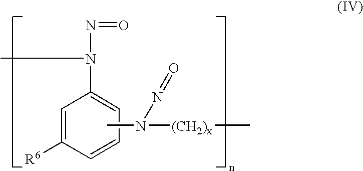

wherein x, n and R⁶ have the same value as described in formula (III).

In another embodiment shown in FIG. 6, the NO-releasing polymer is represented by general formula (V):

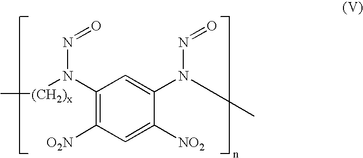

wherein x=2 to 12 and n is an integer greater than about 20.

Formula (VI) represents the polymeric secondary amine precursor to formula (V):

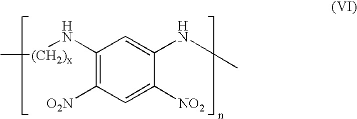

wherein x=2 to 12 and n is an integer greater than about 20. Formula (VI) is described in co-pending application U.S. Ser. No. 11/346,952, “Solvent Resistant Polymers,” Dillip K. Mohanty and Ajit Sharma, inventors.

A suitable primary amine polymer starting material may include, without limitation, a hyperbranched poly(ethyleneimine), a polyvinylamine, or a polyallylamine, as well as a polypropylenimine tetraamine dendrimer or a natural product such as chitosan. The starting primary amine polymer is reacted with a monofluoroaryl, difluoronitroaryl, perfluoroaryl, difluorocyanoaryl, difluoro-trifluoromethylaryl, or difluoro-phenylsulfoxyaryl compound in the presence of anhydrous potassium carbonate using a dipolar aprotic solvent. The product of this reaction yields a secondary amine polymer in accordance with the invention which exhibits highly inert properties. As shown in FIGS. 1-6, the secondary amine, for example, links the nitroaryl group to the polymeric backbone and together forms the repeating unit for the secondary amine polymer.

The secondary amine polymers in accordance with the invention can then undergo a nitrosation reaction to provide the NO-donating polymers, embodying the principles of the invention. As also shown in FIG. 1-6, the secondary amine moiety is converted to an N-nitrosamine group. Upon release of NO, the polymer reverts to the inert secondary amine polymer.

As is demonstrated in the Examples below, the polymers described above were found to be active NO-donating agents and were found to cover a range of release times, which is indicative of the tunability of the polymers to controlled release of NO. Thus, there is potential for a sustained dosing regimen and for systemic mediated activity in accordance with the invention.

The invention is further embodied in soluble, low-molecular weight NO-releasing compounds according to formula (VII):

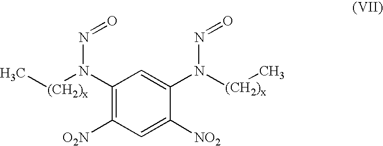

wherein x=5-11.

Compounds of formula (VIII):

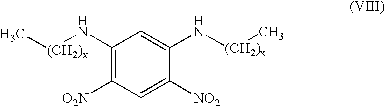

wherein x=5-11, are easily nitrosated to produce the corresponding N-nitrosamines of formula (VII). This mechanism is further illustrated in FIG. 6C.

The NO-donors embodying the principles of the invention may be beneficially used in the treatment of neurodegenerative and other medical conditions associated with NO. Non-limiting examples of medical conditions in which modulating, and suitably elevating, the NO level is beneficial include cardiovascular diseases or disorders, gastrointestinal diseases or disorders, inflammatory diseases or disorders, respiratory diseases or disorders, central nervous system diseases or disorders, neurodegenerative diseases or disorders, psychiatric diseases or disorders, blood pressure-associated diseases or disorders, coronary artery diseases or disorders, atherosclerosis, cholesterol level-associated diseases or disorders, arterial thrombotic diseases or disorders, a heart failure, a stroke, septic shock, NSAID-induced gastric diseases or disorders, inflammatory bowel diseases or disorders, ischemic renal diseases or disorders, peptic ulcer, diabetes, pulmonary hypertension, sickle cell anemia, an asthma, chronic obstructive pulmonary disease, dementia, epilepsy, neuroinflammatory diseases or disorders, trauma, multiple sclerosis, erectile dysfunction, priapism, other male and female sexual dysfunctions, and age-related diseases or disorders.

Pharmaceutical compositions of NO-donors for use in accordance with the invention thus may be formulated in conventional manner by methods known in the art of pharmacy, using one or more pharmaceutically acceptable carriers comprising excipients and auxiliaries, which facilitate processing of the NO-donors into preparations which, can be used pharmaceutically. Proper formulation is dependent upon the route of administration chosen.

Formulations of the NO-donors may conveniently be presented in unit dosage form. The amount of active ingredient, which can be combined with a carrier material to produce a single dosage form, will vary depending upon the subject being treated, and the particular mode of administration. The amount of active ingredient which can be combined with a carrier material to produce a single dosage form will generally be that amount of the polymer which produces a therapeutic effect.

It is also contemplated that the NO-donors may be administered in a fashion where blood levels are sustained. It is contemplated that the NO-donors according to the invention can be administered orally, rectally, intravenously, intraventricularly, topically, intranasally, intraperitoneally, intestinally, parenterally, intraocularly, intradermally, transdermally, subcutaneously, intramuscularly, transmucosally, by inhalation and/or by intrathecal catheter. Suitably, the NO-donors, according to the invention, are administered orally or intravenously, and optionally topically, transdermally, or by inhalation, depending on the condition and the subject being treated.

It is further contemplated that pharmaceutical formulations of the invention may be manufactured by processes well known in the art, e.g., by means of conventional mixing, dissolving, granulating, dragee-making, tablet or pellet-making, emulsifying, encapsulating, entrapping, or lyophilizing processes.

Pharmaceutical compositions suitable for use in context of the invention include compositions wherein active ingredients are contained in an amount effective to achieve the intended purpose. More specifically, a “therapeutically effective amount” means an amount of NO-donors effective to treat, i.e., prevent, alleviate, or ameliorate, symptoms of disease.

For any NO-donors used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to determine useful doses in humans.

It is contemplated that a therapeutically effective amount for the NO-donors of the invention may range between about 35 mg/kg body weight and about 70 mg/kg body weight.

Depending on the severity and responsiveness of the condition to be treated, dosing can also be a single administration of a slow release composition with course of treatment lasting from several days to several weeks or until therapy is effected or diminution of the disease state is achieved. The amount of a composition to be administered will, of course, be dependent on the subject being treated, the severity of the affliction, the manner of administration, as well as the judgment of the prescribing physician.

The NO-donating polymers or low-molecular weight NO-donating compounds of the invention may be further beneficially utilized as active substances in various medical devices. According to an additional aspect of the invention, there is provided a medical device which includes one or more of the NO-donating polymers of the invention, and a delivery system configured for delivering the NO-donating polymers to a bodily site of a subject. It is envisioned that such medical devices may be therefore used for delivering to or applying on a desired bodily site the NO-donating polymers of the invention. The NO-donating polymers can be incorporated in the medical devices either per se or as a part of a pharmaceutical composition, as described hereinabove. As used herein, the phrase “bodily site” includes any organ, tissue, including brain, membrane, cavity, blood vessel, tract, biological surface or muscle, which delivering thereto or applying thereon the polymers of the present invention is beneficial.

The medical devices according to this aspect of the invention can be any medical device known in the art, including those defined and classified, for example, by the FDA, depending on the condition and bodily site being treated. Such medical devices may include, but are not limited to, stents, vascular grafts, pacemaker leads, heart valves, electrodes, sensors, trocars, guide wires, catheters, penile implants, condoms, ocular lenses, sling materials, sutures, wound dressings/bandages, blood collection bags and storage tubes, and tubing used for blood transfusions and hemodialysis.

It is further contemplated that the NO-donors may be conjugated with other bioactive agents or co-administered with other therapeutic agents or drugs, such as, but not limited to, dopamine agonists, decarboxylase inhibitors, and vascular dialators. It is anticipated that NO-donors used in combination with various other therapeutic agents may give rise to a significantly enhanced or synergistic effect, e.g., on neuronal cells, thus providing an increased therapeutic effect. As an increased therapeutic effect is obtained with the above disclosed combinations, utilizing lower concentrations of the therapeutic drugs compared to the treatment regimes in which the drugs are used alone, there is the potential to provide therapy wherein adverse side effects associated with the additional therapeutic agents are considerably reduced than normally observed with the therapeutic agents used alone in larger doses. Further, lowering the incidence of adverse effects may also improve patient compliance, and improve the quality of life of a patient undergoing treatment.

The term “co-administration” is meant to refer to a combination therapy by any administration route in which two or more agents are administered to a patient or subject. Co-administration of agents may also be referred to as combination therapy or combination treatment. The agents may be in the same dosage formulations or separate formulations. For combination treatment with more than one active agent, where the active agents are in separate dosage formulations, the active agents can be administered concurrently, or they each can be administered at separately staggered times. The agents may be administered simultaneously or sequentially (i.e., one agent may directly follow administration of the other) or the agents may be given episodically (i.e., one can be given at one time followed by the other at a later time, e.g., within a week), as long as they are given in a manner sufficient to allow both agents to achieve effective amounts in the bodily site of interest. The agents may also be administered by different routes, e.g., one agent may be administered intravenously while a second agent is administered intramuscularly, intravenously, or orally. Thus, the additional therapeutic agent may be administered prior to, concomitant with, or after administration of the NO-donor.

For combination therapy of the polymers in accordance with the invention with other therapeutic agents, a sustained therapeutic dosing regimen is also contemplated instead of the concentration spike levels resulting from many conventional dosing regimens.

Reference is now made to the following examples, which together with the above descriptions illustrate the invention in a non-limiting fashion.

### EXAMPLES

### Example 1

### General Chemical Syntheses for Preparing NO-Donating Polymer Compounds

The general synthetic pathway for preparing the NO-donating polymer compounds according to the invention is presented in FIGS. 1-6. In general, a desired polymer is first prepared from a corresponding polymeric primary amine or diamine which is reacted with a fluoronitrobenzene, a difluoronitrobenzene, a perfluorobenzene, a difluorocyanobenzene, a difluoro-trifluoromethylbenzene, or a difluoro-phenylsulfoxybenzene to form a secondary polymeric amine in the presence of anhydrous potassium carbonate using a dipolar aprotic solvent, such as dimethylacetamide. As described below, the amine moiety of the latter is then reacted with sodium nitrite in an acidic medium to produce the desired NO-donating polymer according to the invention, i.e., an N-nitrosamine polymer.

### Example 2

### General Nitrosation Procedure

About 500 mg of a polymeric amine was dissolved in 20 mL of sulfuric acid (in some cases, the polymer solution was filtered through glass wool to remove impurities/insoluble particle). The solution was transferred to a 100 mL, 1-necked round-bottomed flask fitted with a glass stopper and a magnetic stir bar, and the vessel was cooled with dry ice/acetone bath to −20° C. Sodium nitrite (8 molar equivalents) was added slowly to the solution and stirred. The temperature was brought to room temperature, and the reaction was continued for 6 hours. The color changed from golden yellow to orange red overnight. The product solution was poured into ice/water mixture (˜500 mL) to precipitate the crude product. The precipitate was green in color. The crude product was collected via vacuum filtration and rinsed with a large amount of water to remove acid residues. The product was then dissolved in methylene chloride and dried over magnesium sulfate, and the solvent was removed with rotorvap to obtain clean product.

### Example 3

### General Procedure for Released NO Measurements

A 35 mL single-necked round-bottomed flask fitted with a magnetic stir bar was charged with 0.0004 moles of the N-nitrosamine polymer, i.e., the NO-releasing polymer, followed by the addition of cadmium shots (1.0 g, 3.0 mm diameter). A solution consisting of 18 mL of water and 2 mL of phosphate buffer solution (PBS) was then added to the reaction vessel. Stirring was initiated immediately. Samples (250 mL) were withdrawn from the reaction vessel at appropriate times and transferred to small centrifuge vials. Deionized water (50 mL) was placed into two well plates followed by the addition of 50 μL of each samples into these two wells. Commercially available Greiss Reagent was used to test for free NO by measuring absorbance of samples at 570 nm.

### Example 4

### Preparation of Polyamine 6 (x=11) of FIG. 6

Polymerization reactions were carried out in a four-necked 100 mL round-bottom flask fitted with an over-head stirrer, a nitrogen inlet, a thermometer, and a dean-stark trap fitted with a condenser. An oil bath was used as the external heat source. A typical procedure for the preparation of polyamine 6 (x=11), from 1,11-diaminoundecane and difluorodinitrobenzene (DFDNB), is provided below. The diamine (1.862 g, 0.01 mol), and DFDNB (2.04 g, 0.10 mol) were accurately weighed and transferred carefully into the reaction vessel. Anhydrous potassium carbonate (2.20 g, 0.016 mol), diphenylsulfone (20 g), and toluene (20 mL) were added to the reaction vessel. The color of the reaction mixture turned deep yellow, and a slight exotherm was observed. The temperature rose to approximately 35° C. The reaction mixture was heated to 60° C., and the reaction was allowed to continue at this temperature for 30 min. The temperature was gradually increased to solvent reflux, and water, the by-product of the reaction, was removed by azeotropic distillation with toluene. Toluene was then removed via the trap, and temperature was increased gradually to 210° C. over a period of 2 hours and held at that temperature for a period of 15-30 mins. The color of the reaction mixture gradually became deep reddish brown, and its viscosity increased. The reaction mixture, while hot, was then poured into rapidly stirring acetone containing acetic acid (20% v/v), and the precipitated polymer was collected by filtration. The polymer was then extracted with a Soxhlet extractor with acetone, water, and acetone, in that order, to remove residual potassium carbonate and diphenyl sulfone. Released NO was measured as described in Example 3.

### Example 5

### Preparation of Polyamine 1 of FIG. 6 with Varying x Values

Preparation of Polyamine 1 (x=7)

A four-necked, 100 mL, round-bottomed flask fitted with an overhead stirrer, a dean-stark trap with an attached condenser, a nitrogen gas inlet, and a thermometer served as the reaction vessel. The reaction vessel was charged with anhydrous potassium carbonate (2.4396 g, 0.018 mol), followed by the addition of diphenylsulfone (18.0124 g, 0.083 mol). Diaminoheptane (0.8146 g, 0.0065 mol), a white crystalline solid, was measured carefully on a Teflon coated weigh pan and transferred to the reaction vessel. DMAC (15 mL) was used to wash the weighing pan, with solvent flowing directly into the reaction vessel. Stirring was initiated. A 1.0277 g (0.0065 mol) sample of 2,4-difluoronitrobenzene was carefully weighed in a one-dram glass vial, followed by dilution with 5 mL of DMAC. The solution was carefully transferred to the reaction vessel. DMAC (10 mL) was used to wash the vial, and transferred to the reaction vessel using a glass funnel. The viscosity of the reaction mixture increased slightly with the addition of the 2,4-difluoronitrobenzene. Toluene (25 mL) was added to the reaction mixture. The reaction vessel was submerged into an external oil bath and was heated to reflux. Water, the by-product of the reaction, was removed via azeotropic distillation with toluene. After the complete removal of water, the temperature of the reaction mixture was allowed to increase to 165° C. by gradual removal of toluene through the dean-stark trap. Reaction color transitioned from bright yellow to bright orange during this time period. The reaction was allowed to continue at this temperature for six hours at which point DMAC was gradually removed through the dean-stark trap. This allowed for an increase in the temperature of the reaction mixture to 195° C., where it was held constant for three hours. A visible rise is viscosity was observed during this time. The external heat source was removed, and the hot reaction mixture was poured into rapidly stirring water (500 mL). The polymer, which precipitated out, was collected by gravity filtration and transferred to a 100 mL flask containing 60 mL of acetone. The polymer was stirred in acetone for two days, followed by isolation via vacuum filtration. The final product, a yellow/brown solid, was dried in a vacuum oven for 24 hours at room temperature. The final yield was 82%. Released NO was measured as described in Example 3.

Preparation of Polyamine 1 (x=11)

A four-necked, 100 mL, round-bottomed flask fitted with an overhead stirrer, a dean-stark trap with an attached condenser, a nitrogen gas inlet and a thermometer served as the reaction vessel. The reaction vessel was charged with anhydrous potassium carbonate (2.7030 g, 0.019 mol). Diaminoundecane (1.6208 g, 0.0087 mol), a white crystalline solid, was measured carefully on a Teflon coated weigh pan and transferred to the reaction vessel. DMAC (15 mL) was used to wash the weighing pan, with solvent flowing directly into the reaction vessel. Stirring was initiated. A 1.38382 g (0.0087 mol) sample of 2,6-difluoronitrobenzene, a slightly yellow liquid, was carefully weighed in a one-dram glass vial, followed by dilution with 5 mL of DMAC. The solution was carefully transferred to the reaction vessel. DMAC (10 mL) was used to wash the vial and transferred to the reaction vessel using a glass funnel. The reaction mixture was bright orange in color at this time. Toluene (25 mL) was added to the reaction mixture. The reaction vessel was submerged into an external oil bath and was heated to reflux. Water, the by-product of the reaction, was removed via azeotropic distillation with toluene. The reaction color transitioned from bright orange to dark red during this time period. The reaction was allowed to continue at this temperature for twenty-four hours. A subtle rise is viscosity was observed during this time. The external heat source was removed and the reaction vessel was allowed to cool to room temperature under the constant purge of nitrogen. The final color of the reaction mixture was dark red. The crude polymer was obtained by precipitation into 500 mL of rapidly stirring water, followed by filtration under reduced pressure. The polymer was transferred to a 100 mL flask, containing 60 mL of methanol. The polymer was stirred in methanol for one day, followed by isolation via vacuum filtration. The final product, a bright red solid, was dried in a vacuum oven for 24 hours at room temperature. The final yield was 65%. Released NO was measured as described in Example 3.

In summary, the invention provides NO-releasing polymers that are tunable in structure to provide a variety of controlled, sustained releases of NO over time. Upon NO release, the NO-donating polymers form inert secondary amines. These secondary amines are also the precursors of the corresponding NO-donating polymers as the amines are readily nitrosated to form the NO-releasing polymers. The invention also provides low molecular weight NO-donating compounds that are generally soluble. These compounds are also interconverted between the NO-releasing compounds and the amine products.

All patents, publications, references, and data cited herein are hereby fully incorporated by reference. In case of conflict between the present disclosure and incorporated patents, publications, references, and data, the present disclosure should control.

We claim:

1. A composition comprising a polymeric secondary amine having formula (I) 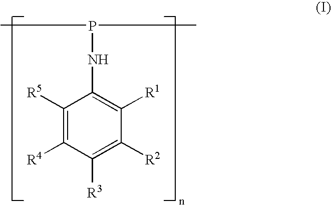
wherein P is a polymeric species with a chemical moiety capable of forming a covalent bond with N, and n is an integer greater than about 20, and R¹, R², R³, R⁴, and R⁵ are H, F, or NO₂ provided that when R¹═F, then R², R³, R⁴, and R⁵═F; and provided that when R¹═NO₂, then R², R³, R⁴, and R⁵═H; provided that when R⁵═NO₂, then R¹, R², R³, and R⁴═H; when R²═NO₂, then R⁴═NO₂ and R¹, R³, and R⁵═H; and when R³═NO₂, then R¹, R², R⁴, and R⁵═H;
or having formula (III) 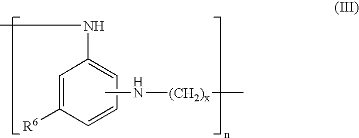
wherein x=2 to 12 and n is an integer greater than about 20, and R⁶ is NO₂, CN, CF₃, or phenyl sulfone;
or a combination thereof.

2. The composition of claim 1, further comprising a bioactive or therapeutic compound.

3. A composition comprising a NO-releasing polymer having formula (II) 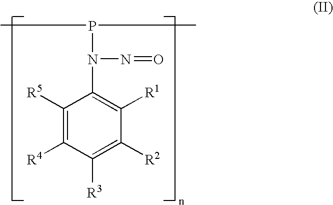
wherein P, n, R¹, R², R³, R⁴, and R⁵ have the same meaning as described above for formula (I);
formula (IV) 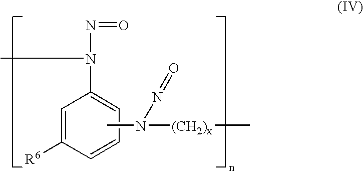
wherein x, n, and R⁶ have the same meaning as described above for formula (III);
formula (V):
or formula (V) 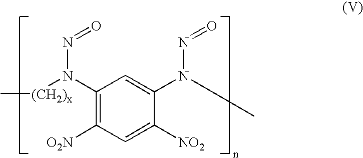
wherein x=2 to 12 and n is an integer greater than about 20;
or a combination thereof.

4. The composition of claim 3, further comprising a bioactive or therapeutic compound.

5. The composition of claim 4, wherein the bioactive agent is chosen from the group consisting of dopamine agonists, decarboxylase inhibitors, and vascular dialators.

6. A composition comprising an insoluble, reactively inert secondary polymeric amine comprising a member of the group consisting of (1) a polymer having at least one pendant secondary amine group covalently bonded to the polymer, the secondary amine substituted on a substituted or unsubstituted aryl group, and (2) a polymer having at least one diamine monomer having the amine groups substituted on a substituted aryl group; and
wherein the substituted aryl group is selected from the group consisting of fluoronitrobenzene, difluoronitrobenzene, perfluorobenzene, difluorocyanobenzene, difluoro-trifluoromethylbenzene, and difluoro-phenylsulfoxybenzene.

7. A composition comprising a NO-releasing polymer comprising a member of the group consisting of (1) a polymer having at least one pendant N-nitrosamine group covalently bonded to the polymer, the N-nitrosamine group substituted on a substituted or unsubstituted aryl group, and (2) a polymer having at least one dinitrosamine monomer having the N-nitosoamine groups substituted on a substituted aryl group.

8. The composition of claim 7, wherein the substituted aryl group is selected from the group consisting of fluoronitrobenzene, difluoronitrobenzene, perfluorobenzene, difluorocyanobenzene, difluoro-trifluoromethylbenzene, and difluoro-phenylsulfoxybenzene.

9. A method of making NO-releasing polymers comprising:
(a) reacting a primary amine polymer with a compound selected from the group comprising monofluoroaryl, difluoronitroaryl, perfluoroaryl, difluorocyanoaryl, difluoro-trifluoromethylaryl, and difluoro-phenylsulfoxyaryl compounds to form a secondary polymeric amine; and
(b) nitrosating the secondary polymeric amine.

10. The method of making NO-releasing polymers of claim 11, further comprising:
(c) storing the NO-releasing polymers at or below about 4° C. in an inert atmosphere.

11. A composition comprising a soluble NO-releasing compound having formula (VII) 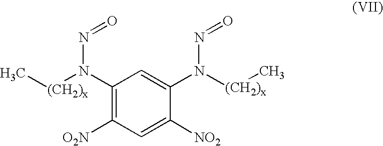
wherein x=5-11.

12. The composition of claim 11, further comprising a bioactive or therapeutic agent.

13. The composition of claim 12, wherein the bioactive agent is selected from the group consisting of dopamine agonists, decarboxylase inhibitors, and vascular dialators.
